(19) United States
(12) Patent Application Publication
(10) Pub. No.: US 2008/0171370 A1
(43) Pub. Date: Jul. 17, 2008

(54) DETOXIFYING PRE-TREATED LIGNOCELLULOSE-CONTAINING MATERIALS

(75) Inventors: Jason W. Holmes, Zebulon, NC (US); Randy Deinhammer, Wake Forest, NC (US); Chee Leong Soong, Raleigh, NC (US)

Correspondence Address: NOVOZYMES NORTH AMERICA, INC., 500 FIFTH AVENUE, SUITE 1600, NEW YORK, NY, 10110, US

(73) Assignee: Novozymes North America, Inc., Franklinton, NC (US)

(21) Appl. No.: 11/954,482

(22) Filed: Dec. 12, 2007

Related U.S. Application Data
(60) Provisional application No. 60/870,420, filed on Dec. 18, 2006.
(60) Provisional application No. 60/890,652, filed on Feb. 20, 2007.

(51) Int. Cl. C12P 7/10; D21C 9/00; C12P 1/00

(52) U.S. Cl. 435/165; 435/277; 435/41

(57) ABSTRACT

The invention relates to a process of detoxifying pretreated lignocellulose-containing maternal by subjecting pre-treated material to a detoxifying compound capable of binding 1) pre-treated lignocellulose degradation products and/or 2) acetic acid. The detoxifying compound may also be an amidase and/or and anhydrase. The invention also relates to a process of producing a fermentation product including a detoxification process of the invention.

### CROSS-REFERENCE TO RELATED APPLICATIONS

This application claims the benefit under 35 U.S.C. 119 of U.S. provisional application Nos. 60/870,420 and 60/890,652 filed Dec. 18, 2006 and Feb. 20, 2007, respectively, the contents of which are fully incorporated herein by reference.

### TECHNICAL FIELD

The present invention relates to processes of detoxifying pre-treated lignocellulose-containing material. The invention also relates to processes of producing a fermentation product from lignocellulose-containing material using a fermenting organism including a detoxification process of the invention.

### BACKGROUND OF THE INVENTION

Due to the limited reserves of fossil fuels and worries about emission of greenhouse gasses there is an increasing focus on using renewable energy sources. Production of fermentation products from lignocellulose-containing material is known in the art and conventionally includes pretreatment, hydrolysis, and fermentation of the lignocellulose-containing material. Pre-treatment results in the release of, e.g., phenolics and furans, from the lignocellulose-containing material that may irreversibly bind enzymes added during hydrolysis and fermentation. These compounds may also be toxic to the fermenting organism's metabolism and inhibit the performance of the fermenting organism.

Detoxification by steam stripping has been suggested but it is a cumbersome and a costly additional process step. It has also been suggested to wash the pre-treated lignocellulose-containing material before hydrolysis. This requires huge amounts of water, that needs to be removed again, and is therefore also costly.

Consequently, there is a need for providing processes for detoxifying pre-treated lignocellulose-containing material suitable for fermentation product production processes.

### SUMMARY OF THE INVENTION

The present invention relates to processes of detoxifying pre-treated lignocellulose-containing material. The invention also relates to processes of producing a fermentation product from lignocellulose-containing material using a fermenting organism including a detoxification process of the invention.

In the first aspect the invention relates to a process of detoxifying pre-treated lignocellulose-containing material, wherein pre-treated lignocellulose-containing material is subjected to one or more compounds selected from the group of:
compound capable of binding pre-treated lignocellulose degradation products, or
compound capable of binding acetic acid; or
amidase; or
anhydrase; or a combination of two of more thereof.

In the second aspect the invention relates to processes of producing a fermentation product from lignocellulose-containing material, comprising the steps of:
(a) pre-treating lignocellulose-containing material;
(b) detoxifying;
(c) hydrolyzing; and
(d) fermenting using a fermenting organism, wherein detoxification is carried out in accordance with a detoxification process of the invention.

### BRIEF DESCRIPTION OF THE FIGURES

FIG. 1 shows amidase and gallic acid dose responses versus control at 24 hours.

FIG. 2 shows the concentration of acetic acid using amidase and gallic acid versus control.

FIG. 3 shows the ethanol concentrations with varying amounts of amidase.

FIG. 4 shows amidase results showing boost in ethanol yield after 24 hours of fermentation.

FIG. 5 shows carbonic anhydrase results showing boost in ethanol production after 12 hours of fermentation.

FIG. 6 shows carbonic anhydrase effect on ethanol production after 24 hours.

### DETAILED DESCRIPTION OF THE INVENTION

In the first aspect the invention relates to processes of detoxifying pre-treated lignocellulose-containing material suitable for producing a fermentation product.

### Lignocellulose-Containing Material

Lignocellulose materials primarily consist of cellulose, hemicellulose, and lignin. The stricture of lignocellulose is not directly accessible to enzymatic hydrolysis. Therefore, the lignocellulose has to be pre-treated, e.g., by acid hydrolysis under adequate conditions of pressure and temperature, in order to break the lignin seal and disrupt the crystalline structure of cellulose. This causes solubilization of the hemicellulose and cellulose fractions. The cellulose fraction can then be hydrolyzed enzymatically, e.g., by cellulolytic enzymes, to convert the carbohydrate polymers into fermentable sugars which may be fermented into a desired fermentation product, such as ethanol. Optionally the fermentation product may be recovered, e.g., by distillation.

Any lignocellulose-containing material is contemplated according to the present invention. The lignocellulose-containing material may be any material containing lignocellulose. In a preferred embodiment the lignocellulose-containing material contains at least 30 wt-%, preferably at least 50 wt.-%, more preferably at least 70 wt-%, even more preferably at least 90 wt-% lignocellulose. It is to be understood that the lignocellulose-containing material may also comprise other constituents such as cellulosic material, including cellulose and hemicellulose, and may also comprise other constituents such as proteinaceous material, starch, sugars, such as fermentable sugars and/or un-fermentable sugars.

Lignocellulose-containing material is generally found, for example, in the stems, leaves, hulls, husks, and cobs of plants or leaves, branches, and wood of trees. Lignocellulose-containing material can also be, but is not limited to, herbaceous material, agricultural residues, forestry residues, municipal solid wastes, waste paper, and pulp and paper mill residues. It is understood herein that lignocellulose-containing material may be in the form of plant cell wall material containing lignin, cellulose, and hemi-cellulose in a mixed matrix.

In a preferred embodiment the lignocellulose-containing material is corn fiber, rice straw, pine wood, wood chips, poplar, bagasse, paper and pulp processing waste.

Other examples include corn stover, hardwood, such as poplar and birch, softwood, cereal straw, such as wheat straw, switchgrass, municipal solid waste (MSW), industrial organic waste, office paper, or mixtures thereof.

In a preferred embodiment the cellulose-containing material is corn stover. In another preferred embodiment the material is corn fiber.

### Process of Detoxifying Pretreated Lignocellulose-Containing Material

When lignocellulose-containing material is pre-treated, degradation products that are toxic to enzymes and fermenting organisms are produced. These toxic compounds severely decrease both the hydrolysis and fermentation rates. Methods for pre-treating lignocellulose-containing material are well known in the art. Examples of contemplated methods are described below in the section “Pre-treatment”.

The present inventors have found that selected compounds can be used to detoxify pre-treated lignocellulose-containing material. These detoxifying compounds are capable of binding pre-treated lignocellulose degradation products and/or acetic acid and can be used to significantly improve the performance of enzymes, e.g., during the hydrolysis step. It was also found that the fermentation time can be reduced as a result of improved performance of the fermenting organism during fermentation. In other words, detoxification carried out in accordance with the invention may result in a shorter “lignocellulose-containing material to fermentation product” process time.

Furthermore, corresponding results may also be obtained by adding amidase and/or anhydrase.

Specific examples of detoxifying compounds can be found below in the “Detoxifying Compounds”-section. In a specific and preferred embodiment the detoxifying compound is gallic acid. Gallic acid was found to be a suitable detoxifying compound for binding phenolics and acetic acid. A plausible theory is that gallic acid is a natural polymer co-monomer; i.e., the core of the gallotannin structure, and therefore is a natural means to polymerize phenolics and also toxins such as acetic acid in a Fischer esterification, e.g., with a sulphuric acid catalyst.

Acid hydrolysis is a commonly used pre-treatment method and therefore these detoxifying compounds can be added during acid hydrolysis so that they are present when the pH is rising for fermentation.

Alternatively, the compound(s), e.g., gallic acid, may also be added in a separate step where the pH is lowered to a suitable pH for the detoxifying compound(s). Afterwards the pH may be adjusted to a pH suitable for fermentation, e.g., a pH below 7.

In the first aspect the invention relates to processes of detoxifying pre-treated lignocellulose-containing material, wherein pre-treated lignocellulose-containing material is subjected to one or more compounds selected from the group of:
compound capable of binding pre-treated lignocellulose degradation products, or
compound capable of binding acetic acid; or
amidase; or
anhydrase; or a combination of two of more thereof.

In an embodiment the detoxifying compound contains radicalizing hydroxyl groups and an esterifiable carboxylic acid group. In a preferred embodiment the compound is gallic acid.

In an embodiment the pretreated lignocellulose degradation products are lignin degradation products and/or hemicellulose degradation products. The pre-treated lignin degradation products may be phenolics in nature.

In another embodiment the hemicellulose degradation product(s) is(are) furans from sugars (such as hexoses and/or pentoses), including xylose, mannose, galactose, rhamanose, and arabinose. Examples of hemicelluloses include xylan, galactoglucomannan, arabinogalactan, arabinoglucuronxylan, glucuronoxylan, and derivatives and combinations thereof.

Examples of inhibitory compounds, i.e., pre-treated lignocellulose degradation products, include 4-OH benzyl alcohol, 4-OH benzaldehyde, 4-OH benzoic acid, trimethyl benzaldehyde, 2-furoic acid, coumaric acid, ferulic acid, phenol, guaiacol, veratrole, pyrogallollol, pyrogallol mono methyl ether, vanillyl alcohol, vanillin, isovanillin, vanillic acid, isovanillic acid, homovanillic acid, veratryl alcohol, veratraldehyde, veratric acid, 2-O-methyl gallic acid, syringyl alcohol, syringaldehyde, syringic acid, trimethyl gallic acid, homocatechol ethyl vanillin, creosol, p-methyl anisol, anisaldehyde, anisic acid, or combinations thereof.

The detoxification process of the invention may preferably be carried out at a pH below 7, preferably below 6. In the case of, e.g., gallic acid a suitable pH would be a pH below 7, preferably below pH 5, especially between pH 1 and 3, such as around pH 2. In a preferred embodiment the temperature during detoxification is a temperature suitable for the detoxifying compound(s). Such suitable temperature can easily be determined by one skilled in the art.

In another embodiment the detoxifying compound is an amidase and/or an anhydrase.

### Amidases

The amidase may be of any origin, especially of microbial original, especially of bacterial or fungal origin.

In preferred embodiments the amidase is selected from the group consisting of: Aminopeptidase B (EC 3.4.11.6) Cytosol alanyl aminopeptidase (EC 3.4.11.14) Dipeptidyl-peptidase II (EC 3.4.14.2), Dipeptidyl-peptidase III (EC 3.4.14.4), Dipeptidyl-peptidase IV (EC 3.4.14.5), Peptidyl-glycinamidase (EC 3.4.19.2), Omega-amidase (EC 3.5.1.3), Amidase (EC 3.5.1.4), Arylformamidase (EC 3.5.1.9), Penicillin amidase (EC 3.5.1.11), Aryl-acytamidase (EC 3.5.1.13), Aminoacylase (EC 3.5.1.14), Nicotinamidase (EC 3.5.1.19), 5-aminopentanamidase (EC 3.5.1.30), Alkylamidase (EC 3.5.1.39), Acylagmatine amidase (EC 3.5.1.40), Formamidase (EC 3.5.1.49), Pentanamidase (EC 3.5.1.50), N-carbamoylputrescine amidase (EC 3.5.1.53), N,N-dimethylformamidase (EC 3.5.1.56), Tryptophanamidase (EC 3.5.1.57), N-carbamoyisarcosine amidase (EC 3.5.1.59), 4-methyleneglutaminase (EC 3.5.1.67), D-benzoylarginine-4-nitroanitide amidase (EC 3.5.1.72), Carnitinamidase (EC 3.5.1.73), Arylalkyl acylamidase (EC 3.5.1.76), Glutathionylspermidine amidase (EC 3.5.1.78). Phthalyl amidase (EC 3.5.1.79), Mandelamide amidase (EC 3.5.1.86), L-lysine-lactamase (EC 3.5.2.11), Phosphoamidase (EC 3.9.1.1), N-sulfoglucosamine sulfohydroase (EC 3.10.1.1), Cyclamate sulfohydrolase (EC 3.10.1.2).

In a preferred embodiment the amidase is an amidase (EC 3.5.1.4).

In a preferred embodiment the amidase is derived from a strain of Pseudomonas, preferably a strain of Pseudomonas aeruginosa.

Amidases may be dosed in the range between 0.01-100 units/g substrate, preferably 0.1-10 units/g substrate, especially 1-5 units/g substrate, such as around 2 units/g substrate or 0.01-1,000 units/g TS (Total Solids), preferably 0.1-500 units/g TS, especially 1-100 units/g TS or from 0.01-100 units/mL, preferably 0.1-50 units/mL, especially 0.2-40 units/mL.

One unit will convert 1.0 mole of acetamide and hydroxylamine to acetohydroxamate and ammonia per min at pH 7.2 at 37° C.

Commercially available amidases include the one from Pseudomonas aeruginosa (Sigma Chemical Co., catalog # A6691).

### Anhydrases

The anhydrase may be of any origin, including of mammal, plant and microbial origin, such as of bacteria and fungal origin. In a preferred embodiment the anhydrase is a carbonic anhydrase classified as EC 4.2.1.1.

Carbonic anhydrases (also termed carbonate dehydratases) catalyze the inter-conversion between carbon dioxide and bicarbonate [CO₂+H₂O⇄HCO₃⁻+H⁴]. An example of a carbonic anhydrase (CA) includes the one discovered in bovine blood (Meidrum and Roughton, 1933, J. Physiol. 80 113-142). Anhydrases are categorzed in three distinct classes called the alpha-, beta- and gamma-class, and potentially a fourth class, the delta-class (Bacteria, Archaea, Sukaryak Tripp et al., 2001 J. Biol. Chem. 276. 48615-48618). For alpha-Cas more than 11 isozymes have been identified in mammals. Alpha-carbonic anhydrases are abundant in all mammalian tissues where they facilitate the removal of CO₂. Beta-Cas are ubiquitous in algae and plants where they provide for CO₂ uptake and fixation for photosynthesis. Gamma-Cas include one from Archaeon Methanosarcina thermophila strain TM-1 (Alber and Ferry, 1994. Proc. Natl. Acad. Sci. USA 91: 6909-6913) and the ones disclosed by Parisi et al., 2004, Plant Mol. Biol. 55; 193-207. In prokaryotes genes encoding all three CA classes have been identified, with the beta- and gamma-class predominating. Many prokaryotes contain carbonic anhydrase genes from more than one class or several genes of the same class (for review see Smith and Ferry, 2000, FEMS Microbiol. Rev 24: 335-366; Tripp et all, 2001, J. Biol. Chem. 276: 48615-48618).

Mammalian-, plant- and prokaryotic carbonic anhydrases (alpha- and beta-class Cas) generally function at physiological temperatures (37° C.) or lower temperatures.

In a preferred embodiment the carbonic anhydrase is one of two heat-stable carbonic anhydrases, namely the beta-class CA (Cab) from Methanobacterium thermoautotrophicum ΔH (Smith and Ferry, 1999, J. Bacteriol. 181: 6247-6253) or the gamma-class carbonic anhydrase (Cam) from Methanosarcina thermophila TM-1 (Alber and Ferry, 1994, Proc. Natl. Acad. Sci. USA 91: 6909-6913; Alber and Ferry, 1996, J. Bacteriol. 178: 3270-3274).

Other examples of carbonic anhydrases include the heat-stable carbonic anhydrases disclosed as SEQ ID NO: 2, SEQ ID NO: 4, SEQ ID NO: 6, SEQ ID NO: 8, SEQ ID NO: 10 or SEQ ID NO: 12 or from Bacillus clausii KSM-K16 (NCBI acc. No. Q5WD44 or SEQ ID NO: 14) or from Bacillus halodurans (NCBI acc. No. Q9KFW1 or SEQ ID NO: 16 in U.S. application No. 60/887,386 (from Novozymes, which are incorporated by reference). In one embodiment the carbonic anhydrase is derived from a strain of Aspergillus ficuum.

In another embodiment the carbonic anhydrase is derived from Bacillus sp. P203 deposited under accession # DSM 19153. The Bacillus sp. P203 carbonic anhydrase is disclosed and concerned in SEQ ID NO: 4 and Examples 8-10 in WO 2007/019859 (Novozymes A/S) which is hereby incorporated by reference.

Anhydrase or carbonic anhydrase may be dosed in the range between 0.01-1,000 kilo units/mL, preferably 0.1-500 kilo units/mL, especially 0.2-400 kilo units/mL or 0.01-1,000 kilo units/g TS (Total Solids), preferably 0.1-500 kilo units/g TS, especially 0.2-400 kilo units/g TS.

Commercially available anhydrases include a carbonic anhydrase from bovine erythrocytes (Sigma Chemical Co., catalog # A3934).

Production of Fermentation Products from Lignocellulose-Containing Material

In the second aspect the invention relates to processes of producing a fermentation product from lignocellulose-containing material.

More precisely the invention relates to processes of producing a fermentation product from lignocellulose-containing material, comprising the steps of:
(a) pre-treating lignocellulose-containing material;
(b) detoxifying;
(c) hydrolyzing; and
(d) fermenting using a fermenting organism, wherein detoxification is carried out in accordance with a detoxification process of the invention.

According to the invention one or more detoxifying compounds is(are) added to the pre-treated lignocellulosic material in step (b). The detoxification step (b) and the hydrolysis step (c) may be carried out simultaneously or sequentially.

According to the invention hydrolysis step (c) and fermentation step (d) may be carried out sequentially or simultaneously. Therefore, the pre-treated lignocellulose-containing material may be hydrolyzed before fermentation or carried out as simultaneous hydrolysis and fermentation (SHF or SHHF). In a further embodiment steps (c) and (d) are carried out as hybrid hydrolysis and fermentation (HHF).

Simultaneous hydrolysis and fermentation (SHF) in general means that hydrolysis and fermentation are combined and carried out at conditions (e.g., temperature and/or pH) suitable for the fermenting organism in question.

Hybrid hydrolysis and fermentation (HHF) begins with a separate hydrolysis step and ends with a simultaneous hydrolysis and fermentation step (SHF). The separate hydrolysis step is an enzymatic cellulose saccharification step typically carried out at conditions (e.g., at higher temperature) suitable, preferably optimal, for the hydrolyzing enzyme(s) in question. The subsequent simultaneous hydrolysis and fermentation step (SHF) is typically carried out at conditions suitable for the fermenting organism (often at lower temperature than the separate hydrolysis step).

If the pre-treated cellulose-containing material is hydrolyzed enzymatically, it is advantageous to do detoxification before and/or simultaneously with hydrolysis. However, if hydrolysis is carried out using one or more acids, i.e., acid hydrolysis detoxification is preferably carried out after and/or simultaneously with acid hydrolysis.

In another embodiment detoxification step (b) may be carried separately from hydrolysis step (c) and fermentation step (d) which may be carried out simultaneously. In a further embodiment all of steps (b), (c) and (d) are carried out simultaneously or sequentially.

### Detoxifying Compounds

According to the invention detoxifying compounds may be compounds selected from the group of compounds capable of binding pre-treated lignocellulose degradation products, or compounds capable of binding acetic acid, or amidase: and/or anhydrase. The compounds may be used alone or in combination of two of more thereof.

Examples of amidases and anhydrases can be found above in the “Amidases” and “Anhydrases”-sections.

Examples of detoxifying compounds include p-hydroxy benzaldehyde, p-hydroxy benzoic acid, p-coumaric acid, anisaldehyde, anisic acid, catechol, salicylic acid, m-hydroxy benzoic acid, protocatecualdehyde, protocatuic acid, isovanillic acid, vanillin, vanillyl alcohol, vanillic acid, coniferyl alcohol, ferulic acid, guaiacyl glycerol, veratraldehyde, veratric acid, gentisic acid, syringaldehyde, syringic acid and gallic acid.

It should be understood that the detoxifying compound(s) should preferably be present together with a catalyst to bind and/or polymerize the toxic compound(s). The catalyst would ideally be sulphuric acid, but could also be a Lewis acid. The pH should be brought to a pH level that results in an environment that is suitable for a Fischer esterification to occur. A person skilled in the art would be able to determine suitable catalysts and conditions, e.g., pH conditions which would be different for different substrates. For instance, for corn stover a pH between 1 and 3, preferably around 2 would be suitable.

What an effect dose/concentration of a detoxifying compound is depends not only on the compounds in question, but also on process conditions and the catalyst. It should be noted that compounds, such as garlic acid, which has an inhibitory effect when present in the liquor coming from pre-treatment, may when used in an effective dose/concentration and subjected to a suitable catalyst under suitable conditions function as a detoxifying compound.

One skilled in the art can easily determine what an effective dose/concentration of a detoxifying compound is.

In a preferred embodiment the detoxifying compound used is gallic acid.

The detoxifying compound(s), preferably gallic acid, may be added to either washed and/or unwashed lignocellulose-containing material before, during and/or after pre-treatment in step (a). In a preferred embodiment the pre-treated lignocellulose-containing material is unwashed.

Gallic acid has three hydroxyl groups for forming acetyl-esters which in turn can occupy the inhibitory effect of acetic acid. Gallic acid takes no part in the actual fermentation.

In addition the carboxylic acid group of gallic acid can react with phenolic compounds from lignin and/or its degradation products.

In general esterification can be maintained when the pH stays below neutral (around pH 7), preferably below pH 6. In an embodiment gallic acid is recycled when the pH is driven to slightly alkaline conditions, thus reducing the acetyl ester to acetic acid and returning the gallic acid to its native state.

In an embodiment the detoxifying compound(s) is(are), preferably gallic acid, is(are) dosed in a concentration of below 1000 mM, such as between 0.001-1000 mM, preferably below 100 mM, such as between 0.001-100 mM, more preferably below 10 mM, such as between 0.001-10 mM, or especially below 1 mM, such as between 0.001-1 mM.

### Pre-Treatment

The lignocellulose-containing material is pre-treated in step (a) before being hydrolyzed and fermented sequentially or simultaneously. The goal of pre-treatment is to separate and/or release cellulose, hemicellulose and/or lignin and this way improve the rate of enzymatic hydrolysis. Pre-treatment methods such as wet-oxidation and alkaline pre-treatment targets lignin, while dilute acid and auto-hydrolysis targets hemicellulose. Steam explosion is an example of a pre-treatment that targets cellulose.

According to the invention pre-treatment step (a) may be a conventional pre-treatment step known in the art. Pre-treatment may take place in aqueous slurry. The lignocellulose-containing material may during pre-treatment be present in an amount between 10-80 wt.-%, preferably between 20-70 wt.-%, especially between 30-60 wt.-%, such as around 50 wt-%.

### Chemical and Mechanical

The lignocellulose-containing material may according to the invention be chemically and/or mechanically pre-treated before hydrolysis and/or fermentation. Mechanical treatment (often referred to as physical treatment) may be used alone or in combination with subsequent or simultaneous hydrolysis, especially enzymatic hydrolysis, to promote the separation and/or release of cellulose, hemicellulose and/or lignin.

The chemical and/or mechanical pretreatment may be carried out prior to hydrolysis and/or fermentation. Alternatively, chemical and/or mechanical is carried out simultaneously with hydrolysis, such as simultaneously with addition of one or more cellulolytic enzymes, or other enzyme activities mentioned below, to release fermentable sugars, such as glucose and/or maltose.

In an embodiment of the invention the pre-treated lignocellulose-containing material is washed before detoxification in step (b). Washing may improve the fermentability of, e.g., dilute-acid hydrolyzed lignocellulose-containing material, such as, e.g., corn stover.

### Chemical Pre-Treatment

The term “chemical treatment” refers to any chemical pretreatment which promotes the separation and/or release of cellulose, hemicellulose and/or lignin. Examples of suitable chemical pre-treatments include treatment with for example, dilute acid. Further, wet oxidation is also considered chemical pre-treatment.

Preferably, the chemical pretreatment is acid treatment, more preferably, a continuous dilute and/or mild acid treatment, such as, treatment with sulphuric acid, or another organic acid, such as acetic acid, citric acid, tartaric acid, succinic acid, or mixtures thereof. Other acids may also be used. Mild acid treatment means in the context of the present invention that the treatment pH lies in the range from 1-5, preferably 1-3. In a specific embodiment the acid concentration is in the range from 0.1 to 2.0 wt % acid, preferably sulphuric acid. The acid may be mixed or contacted with the material to be fermented according to the invention and the mixture may be held at a temperature in the range of 160-220° C., such as 165-195° C., for periods ranging from minutes to seconds, e.g., 1-60 minutes, such as 2-30 minutes or 3-12 minutes. Addition of strong acids, such as sulphuric acid, may be applied to remove hemicellulose. This enhances the digestibility of cellulose.

Wet oxidation techniques involve use of oxidizing agents, such as: sulphite based oxidizing agents or the like. Examples of solvent pre-treatments include treatment with DMSO (Dimethyl Sulfoxide) or the like. Chemical pretreatment is generally carried out for 1 to 60 minutes, such as from 5 to 30 minutes, but may be carried out for shorter or longer periods of time dependent on the material to be pretreated.

### Mechanical Pre-Treatment

As used in context of the present invention the term “mechanical pretreatment” refers to any mechanical (or physical) treatment which promotes the separation and/or release of cellulose, hemicellulose and/or lignin from lignocellulose-containing material. For example, mechanical pre-treatment includes various types of milling, irradiation, steaming/steam explosion, and hydrothermolysis.

Mechanical pre-treatment includes comminution (mechanical reduction of the particle size). Comminution includes dry milling, wet milling and vibratory ball milling. Mechanical pretreatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is a batch-process, steam gun hydrolyzer system which uses high pressure and high temperature as defined above. A Sunds Hydrolyzer (available from Sunds Defibrator AB (Sweden)) may be used for this.

### Combined Chemical and Mechanical Pre-Treatment

In a preferred embodiment both chemical and mechanical pre-treatment are carried out involving, for example, both dilute or mild acid treatment and high temperature and pressure treatment. The chemical and mechanical pre-treatment may be carried out sequentially or simultaneously, as desired.

Accordingly, in a preferred embodiment, the lignocellulose-containing material is subjected to both chemical and mechanical pre-treatment to promote the separation and/or release of cellulose, hemicellulose and/or lignin.

In a preferred embodiment the pre-treatment is carried out as a dilute and/or mild acid steam explosion step.

### Hydrolysis

Before and/or simultaneously with fermentation the pre-treated lignocellulose-containing material may be hydrolyzed in order to break the lignin seal and disrupt the crystalline structure of cellulose. The dry solids content during hydrolysis may be in the range from 5-50 wt-%, preferably 10-40 wt-%, preferably 20-30 wt-%. Hydrolysis may be carried out as a fed batch process where the pre-treated lignocellulose-containing material (substrate) is fed gradually to an, e.g., enzyme containing hydrolysis solution.

In an embodiment of the invention detoxification takes place before or during hydrolysis.

In a preferred embodiment hydrolysis is carried out enzymatically. According to the F invention the pretreated lignocellulose-containing material, may be hydrolyzed by one or more hydrolases (class EC 3 according to the Enzyme Nomenclature), preferably one or more carbohydrases selected from the group consisting of cellulase, hemicellulase, amylase, such as alpha-amylase, carbohydrate-generating enzyme, such as glucoamylase. A protease may also be present. Alpha-amylase, glucoamylase and/or the like may be present during hydrolysis and/or fermentation as the lignocellulose-containing starting material may include some starch.

The enzyme(s) used for hydrolysis is(are) capable of directly or indirectly converting carbohydrate polymers into fermentable sugars which can be fermented into a desired fermentation product, such as ethanol.

In a preferred embodiment the carbohydrase has cellulolytic enzyme activity. Suitable carbohydrases are described in the “Enzymes”-section below,.

Hemicellulose polymers can be broken down by hemicelluloses and/or acid hydrolysis to release its five and six carbon sugar components. The six carbon sugars (hexoses), such as glucose, galactose, arabinose and mannose, can readily be fermented to, e.g., ethanol, acetone, butanol, glycerol, citric acid, fumaric acid etc. by suitable fermenting organisms including yeast. Preferred for ethanol fermentation is yeast of the species Saccharomyces cerevisiae, preferably strains which are resistant towards high levels of ethanol, i.e., up to, e.g., about 10, 12 or 15 vol. % or more ethanol such as 20 vol. %.

In a preferred embodiment the pre-treated lignocellulose-containing material is hydrolyzed using a hemicellulase, preferably a xylanase, esterase, cellobiase, or combination thereof.

Hydrolysis may also be carried out in the presence of a combination of hemicelluloses and/or cellulases, and optionally one or more of the other enzyme activities mentioned in the “Enzyme” section below.

Enzymatic treatment may be carried out in a suitable aqueous environment under conditions which can readily be determined by one skilled in the art. In a preferred embodiment hydrolysis is carried out at optimal conditions for the enzyme(s) in question,

Suitable process time, temperature and pH conditions can readily be determined by one skilled in the art present invention. Preferably, hydrolysis is carried out at a temperature between 25 and 70° C., preferably between 40 and 60° C., especially around 50° C. The process is preferably carried out at a pH in the range from 3-8, preferably pH 4-6, especially around pH 5. Preferably, hydrolysis is carded out for between 8 and 72 hours, preferably between 12 and 48 hours, especially around 24 hours.

According to the invention hydrolysis in step (b) and fermentation in step (c) may be carried out simultaneously (SHF process) or sequentially (HHF process).

### Fermentation

According to the invention the pre-treated (and hydrolyzed) lignocellulose-containing material is fermented by at least one fermenting organism capable of fermenting fermentable sugars, such as glucose, xylose, mannose, galactose, and/or arabinose, directly or indirectly into a desired fermentation product.

The fermentation is preferably ongoing for 24 to 96 hours, in particular 35 to 60 hours.

In an embodiment the fermentation is carried out at a temperature between 20 to 40° C., preferably 26 to 34° C., in particular around 32° C. In an embodiment the pH is from pH 3 to 6, preferably around pH 4 to 5.

Contemplated is a simultaneous hydrolysis and fermentation (SHF) where there is no separate holding stage for the hydrolysis, meaning that the hydrolyzing enzyme(s) and the fermenting organism are added together. When the fermentation is performed simultaneous with hydrolysis the temperature is preferably between 30° C. and 35° C., and more preferably between 31° C. and 34° C., such as around 32° C. A temperature program comprising at least two holding stages at different temperatures may be applied according to the invention.

The process of the invention may be performed as a batch or as a continuous process.

### Recovery

Subsequent to fermentation the fermentation product may be separated from the fermentation broth. The broth may be distilled to extract the fermentation product or the fermentation product may be extracted from the fermentation broth by micro or membrane filtration techniques. Alternatively the fermentation product may be recovered by stripping. Recovery methods are well known in the art.

### Fermentation Products

The process of the invention may be used for producing any fermentation product. Especially contemplated fermentation products include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B12, beta-carotene); and hormones.

Also contemplated products include consumable alcohol industry products, e.g., beer and wine; dairy industry products, e.g., fermented dairy products; leather industry products and tobacco industry products. In a preferred embodiment the fermentation product is an alcohol, especially ethanol. The fermentation product, such as ethanol, obtained according to the invention, may preferably be used as fuel. However, in the case of ethanol it may also be used as potable ethanol.

### Fermenting Organism

The term “fermenting organism” refers to any organism, including bacterial and fungal organisms, suitable for producing a desired fermentation product. Especially suitable fermenting organisms according to the invention are able to ferment, i.e., convert, sugars, such as glucose, directly or indirectly into the desired fermentation product. Examples of fermenting organisms include fungal organisms, such as yeast. Preferred yeast includes strains of the genus Saccharomyces, in particular a strain of Saccharomyces cerevisiae or Saccharomyces uvarum: a strain of Pichia, in particular Pichia stipitis or Pichia pastoris; a strain of the genus Candida, in particular a strain of Candida utilis, Candida arabinofermentans, Candida diddensii, Candida sonorensis, Candida skehatae, Candida tropcalis, or Candida boidinii. Other contemplated yeast includes strains of Hansenula, in particular Hansenula polymorpha or Hansenula anomala; strains of Kluyveromyces in particular Kluyveromyces marxianus or Kluyveromyces fagilis, and strains of Schizosaccharomyces, in particular Schizosaccharomyces pombe.

Preferred bacterial fermenting organisms include strains of Escherichia, in particular Escherichia coli, strains of Zymomonas in particular Zymomonas mobilis, strains of Zymobacter in particular Zymobactor palmae, strains of Klebsiella in particular Klebsiella oxytoca, strains of Leuconostoc in particular Leuconostoc mesenteroides, strains of Clostridium in particular Clostridium butyricum, strains of Enterobacter in particular Enterobacter aerogenes and strains of Thermoanaerobacter, in particular Thermoanaerobacter BG1L1 (Appl. Micro. Biotech. 77; 61-86) and Thermoanarobacter ethanolicusm, Thermoanaerobacter thermosaccharolyticum, or Thermoanaerobacter mathranii. Strains of Lactobacillus are also envisioned as are strains of Corynebacterium glutamicum R, Bacillis thermoglucosidaisus, and Geobacillus thermoglucosidasius.

In an embodiment the fermenting organism is a C6 sugar fermenting organism, such as a strain of e.g., Saccharomyces cerevisiae.

In connection with especially fermentation of lignocellulose derived materials C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp that are capable of fermenting C5 sugars include the ones concerned in, e.g., Ho et al., 1998, Applied and Environmental Microbiology, p. 1852-1859 and Karhumaa et al., 2006, Microbial Cell Factories 5:18.

In one embodiment the fermenting organism is added to the fermentation medium so that the viable fermenting organism, such as yeast, count per mL of fermentation medium is in the range from 10⁵ to 10¹², preferably from 10⁷ to 10¹0, especially about 5×10⁷.

Commercially available yeast includes, e.g., RED START™, and ETHANOL RED™ yeast (available from Fermentis/Lesaffre, USA), FALI (available from Fleischmann's Yeast, USA), SUPERSTART and THERMOSACC™ fresh yeast (available from Ethanol Technology, Wis., USA), BIOFERM AFT and XR (available from NABC—North American Bioproducts Corporation, Ga., USA), GERT STRAND (available from Geta Strand AB, Sweden), and FERMIOL (available from DSM Specialties).

### Enzymes

Even though not specifically mentioned in context of a process of the invention, it is to be understood that the enzyme(s) is(are) used in an “effective amount”.

### Cellulases or Cellulolytic Activity

The term “cellulolytic activity” or “cellulase activity” as used herein are understood as comprising enzymes having cellobiohydrolase activity (EC 3.2.1.91), e.g., cellobiohydrolase I and/or cellobiohydrolase II, as well as endo-glucanase activity (EC 3.2.1.4) and/or beta-glucosidase activity (EC 3.2.1.21). See relevant sections below with further description of such enzymes.

At least three categories of enzymes are important for converting cellulose into fermentable sugars: endoglucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases seem to be the key enzymes for degrading native crystalline cellulose.

The cellulolytic activity may, in a preferred embodiment, be in the form of a preparation of enzymes of fungal origin, such as from a strain of the genus Trichoderma: preferably a strain of Trichoderma reesei; a strain of the genus Humicola: such as a strain of Humicola insolens; or a strain of Chrysosporium, preferably a strain of Chrysosporium lucknowense.

In preferred embodiment the cellulolytic enzyme preparation contains one or more of the following activities: cellulase, hemicellulase, cellutolytic enzyme enhancing activity, beta-glucosidase activity, endogtucanase, or cellubiohydrolase.

In a preferred embodiment cellulolytic enzyme preparation is a composition concerned in U.S. application No. 60/941,251, which is hereby incorporated by reference.

In a preferred embodiment the cellulolytic enzyme preparation comprising a polypeptide having cellutolytic enhancing activity, preferably a family GH61A polypeptide, preferably those disclosed in WO 2005/074656 (Novozymes). The cellulolytic enzyme preparation may further comprise beta-glucosidase,, such as beta-glucosidase derived from a strain of the genus Trichoderma, Aspergillus or Penicillium, including the fusion protein having beta-glucosidase activity disclosed in U.S. application No. 60/832,511 or U.S. application Ser. No. 11/781,151 (Novozymes). In a preferred embodiment the cellulolytic enzyme preparation may also comprises a CBH II enzyme, preferably Thielavia terrestris cellobiohydrolase II (CEL6A). In another embodiment the cellutolytic enzyme preparation may also comprise cellutolytic enzymes; preferably those derived from Trichoderma reesei or Humicola insolens.

The cellulolytic enzyme preparation may also comprising a polypeptide having cellulolytic enhancing activity (GH61A) disclosed in WO 2005/074656; a cellobiohydrolase, such as Thielavia terrestis cellobiohydrolase II (CEL6A), a beta-glucosidase (e.g., the fusion protein disclosed in U.S. application No. 60/832,511) and cellulolytic enzymes, e.g., derived from Trichoderma reesei.

In a preferred embodiment the cellutolytic composition comprising a polypeptide having cellulolytic enhancing activity (GH61A) disclosed in WO 2005/074656; a beta-glucosidase (e.g., the fusion protein disclosed in U.S. application No. 60/832,511 or 11/781,151), and cellutolytic enzymes preparation, e.g., derived from Trichoderma reesei.

In another preferred embodiment the cellulolytic composition comprising a polypeptide having cellulolytic enhancing activity (GH61A) disclosed in WO 2005/074656;: a beta-glucosidase (fusion protein disclosed in U.S. application No. 60/832,511), and cellulolytic enzymes preparation derived from Trichoderma reesei.

In an embodiment the cellulolytic enzyme composition is the commercially available product CELLUCLAST™ 1.5L or CELLUZYME™ (Novozymes A/S, Denmark).

The cellutolytic or cellulase activity may be dosed in the range from 0.1-100 FPU per gram total solids (TS), preferably 0.550 FPU per gram TS, especially 1-20 FPU per gram TS.

### Endoglucanase (EG)

The term “endoglucanase” means an endo-1,4-(1,3;1,4)-beta-D-glucan 4-glucanohydrolase (E.C. No. 3.2.1.4), which catalyses endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity may be determined using carboxymethyl cellulose (CMC) hydrolysis according to the procedure of Ghose, 1987, Pure and Appl. Chem. 59: 257-268.

In a preferred embodiment endoglucanases may be derived from a strain of the genus Trichoderma, preferably a strain of Trichoderma reesei; a strain of the genus Humicola, such as a strain of Humicola insolens, or a strain of Chrysosporium, preferably a strain of Chrysosporium lucknowense.

### Cellobiohydrolase (CBH)

The term “cellobiohydrolase” means a 1,4-beta-D-glucan cellobiohydrolase (E.G. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydrolases are mentioned above including CBH I and CBH II from Trichoderma reseei; Humicola insolens and CBH II from Thielavia terrestris cellobiohydrolase (CEL6A).

Cellobiohydrolase activity may be determined according to the procedures described by Lever et at,, 1972, Anal. Biochem. 47: 273-279 and by van Tilbeurgh et al., 1982, FEBS Letters 149: 152-156, van Tilbeurgh and Claeyssens, 1985., FEBS Letters 187: 283-288. The Lever et at. method is suitable for assessing hydrolysis of cellulose in corn stover and the method of van Tilbeurgh et at is suitable for determining the cellobiohydrolase activity on a fluorescent disaccharide derivative.

### Beta-Glucosidase

One or more beta-glucosidases or “cellobiase” may be present for hydrolysis.

The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, J. Basic Microbiol. 42; 55-66, except different conditions were employed as described herein. One unit of beta-glucosidase activity is defined as 1.0 micro mole of p-nitrophenol produced per minute at 50° C., pH 5 from 4 mM p-nitrophenyl-beta-D-glucopyranoside as substrate in 100 mM sodium citrate, 0.01% TVWEN® 20.

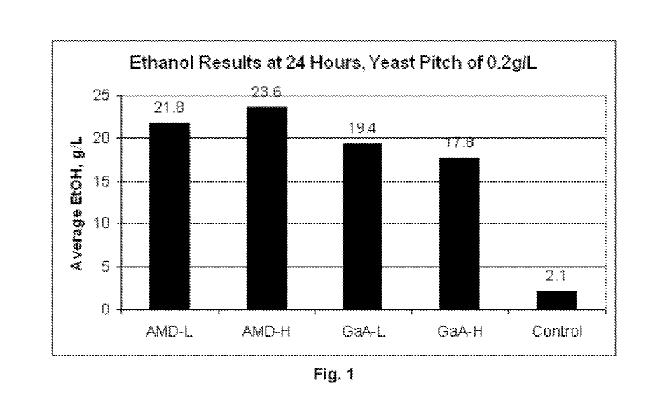

In a preferred embodiment the beta-glucosidase is of fungal origin, such as a strain of the genus Trichoderma. Aspergillus or Penicillium. In a preferred embodiment the beta-glucosidase is a derived from Trichoderma reesei, such as the beta-glucosidase encoded by the bgl1 gene (see FIG. 1 of EP 562003). In another preferred embodiment the beta-glucosidase is derived from Aspergillus oryzae (recombinantly produced in Aspergillus oryzae according to WO 02/095014), Aspergillus fumigatus (recombinantly produced in Aspergillus oryzae according to Example 22 of WO 02/095014) or Aspergillus niger (see, e.g., 1981, J. Appl. 3: 157-163).

### Hemicellulolytic Enzymes

According to the invention the lignocellulose-containing material may further be subjected to one or more hemicellulolytic enzymes, e.g., one or more hemicellulases.

Hemicellulose can be broken down by hemicellulases and/or acid hydrolysis to release its five and six carbon sugar components.

In an embodiment of the invention the lignocellulose derived material may be treated with one or more hemicellulases.

Any hemicellulase suitable for use in hydrolyzing hemicellulose, preferably into xylose, may be used. Preferred hemicellulases include xylanases, arabinofuranosidases, acetyl xylan esterase, feruloyl esterase, glucuronidases, endo-galactanase, mannases, endo or exo arabinases, exo-galactanses, pectinases, xyloglucanases, and mixtures of two or more thereof.

Preferably, the hemicellulase for use in the present invention is an exo-acting hemicellulase, and more preferably, the hemicellulase is an exo-acting hemicellulase which has the ability to hydrolyze hemicellulose under acid conditions of below pH 7, preferably pH 3-7. An example of hemicellulase suitable for use in the present invention includes VISCOZYME™ (available from Novozymes A/S, Denmark).

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus such as Aspergillus aculeatus; or a strain of Humicola, preferably Humicola langinosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes A/S, Denmark.

Arabinofuranosidases (EC 3.2.1.55) catalyze the hydrolysis of terminal non-reducing alpha-L-arabinofuranoside residues in alpha-L-arabinosides.

Galactanases (EC 3.2.1.89), arabinogalactan endo-1,4-beta-galactosidases, catalyze the endohydrolysis of 1,4-D-galactosidic linkages in arabinogalactans.

Pectinases (EC 3.2.1.15) catalyze the hydrolysis of 1,4-alpha-D-galactosiduronic linkages in pectate and other galacturonans.

Xyloglucanases catalyze the hydrolysis of xyloglucan.

The hemicellulase may be added in an amount effective to hydrolyze hemicellulose, such as, in amounts from about 0.001 to 0.5 wt.-% of total solids (TS), more preferably from about 0.05 to 0.5 wt.-% of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate.

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose-containing material by cellulolytic protein under the following conditions: 1-50 mg of total protein/g of cellulose in PCS (pre-treated corn stover), wherein total protein is comprised of 80-99.5% w/w cellulolytic protein/g of cellulose in PCs and 0.5-20% w/w protein of cellulolytic enhancing activity for 1-7 day at 50° C. compared to a control hydrolysis with equal total protein loading without cellutolytic enhancing activity (1-50 mg of cellulolytic protein/g of cellulose in PCs).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellutolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Application Publication No. 2007/0077630 discloses an isolated polypeptide having cellulolytic enhancing activity and a polynucleotide thereof from Trichoderma reesei.

A cellulolytic enzyme may be added for hydrolyzing the pre-treated lignocellulose-containing material. The cellulase may be dosed in the range from 0.1-100 FPU per gram total solids (TS), preferably 0.5-50 FPU per gram TS, especially 1-20 FPU per gram TS.

### Alpha-Amylase

According to the invention an alpha-amylase may be used. In a preferred embodiment the alpha-amylase is an acid alpha-amylase, e.g., fungal acid alpha-amylase or bacterial acid alpha-amylase. The term “acid alpha-amylase” means an alpha-amylase (E.G. 3.2.1.1) which added in an effective amount has activity optimum at a pH in the range of 3 to 7, preferably from 3.5 to 6, or more preferably in the range from pH 5-6.

### Bacterial Alpha-Amylase

According to the invention the bacterial alpha-amylase is preferably derived from the genus Bacillus.

In a preferred embodiment the Bacillus alpha-amylase is derived from a strain of B. licheniformis, B. amyloliquefaciens, B. subtilis or B. stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/119467 (all sequences hereby incorporated by reference). In an embodiment of the invention the alpha-amylase may be an enzyme having a degree of identity of at least 60%. preferably at least 70%, more preferred at least 80%, even more preferred at least 90%, such as at least 95%, at least 96%, at least 97%, at least 98% or at least 99% to any of the sequences shown in SEQ ID NO: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 and 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta (181-182) compared to the wild-type BSG alpha-amylase amino acid sequence set forth in SEQ ID NO: 3 disclosed in WO 99/19467 or deletion of amino acids R179 and G180 using SEQ ID NO: 3 in WO 99/19467 for numbering (which reference is hereby incorporated by reference). Even more preferred are Bacillus alpha-amylases, especially Bacillus stearothermophilus alpha-amylase, which have a double deletion corresponding to delta (181-182) and further comprise a N193F substitution (also denoted I181*+G182*+N193F) compared to the wild-type BSG alpha-amylase amino acid sequence set forth in SEQ ID NO: 3 disclosed in WO 99/19467.

### Bacterial Hybrid-Alpha-Amylase

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitution:

G48A+T491+G107A+H156Y+A181T+N190F+1201F+A209V+Q264S (using the Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using SEQ ID NO: 5 numbering of WO 99/19467).

### Fungal Alpha-Amylase

Fungal alpha-amylases include alpha-amylases derived from a strain of the genus Aspergillus, such as, Aspergillus oryzae, Aspergillus niger and Aspergillus kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e., more than 70%, more than 75%, more than 80%, more than 85% more than 90%, more than 95%, more than 96%, more than 97%, more than 98%, more than 99% or even 100% identity to the mature part of the amino acid sequence shown in SEQ ID NO: 10 in WO 96/23874.

Another preferred acidic alpha-amylase is derived from a strain Aspergillus niger. In a preferred embodiment the acid fungal alpha-amylase is the one from A. niger disclosed as “AMYA_ASPNG” in the Swiss-prot/TeEMBL database under the primary accession no. P56271 and described in WO 89/01969 (Example 3). A commercially available acid fungal alpha-amylase derived from Aspergillus niger is SP288 (available from Novozymes A/S, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al., 1996, J. Ferment. Bioeng. 81:292-298, “Molecular-cloning and determination of the nucleotide-sequence of a gene encoding an acid-stable alpha-amylase from Aspergillus kawachii”, and further as EMBL:#AB008370.

The fungal alpha-amylase may also be a wild-type enzyme comprising a starch-binding domain (SBD) and an alpha-amylase catalytic domain (i.e., non-hybrid), or a variant thereof. In an embodiment the wild-type alpha-amylase is derived from a strain of Aspergillus kawachii.

### Fungal Hybrid Alpha-Amylase

In a preferred embodiment the fungal acid alpha-amylase is a hybrid alpha-amylase. Preferred examples of fungal hybrid alpha-amylases include the ones disclosed in WO 2005/003311 or U.S. Application Publication no. 2005/0054071 (Novozymes) or U.S. application No. 60/638,614 (Novozymes) which is hereby incorporated by reference. A hybrid alpha-amylase may comprise an alpha-amylase catalytic domain (CD) and a carbohydrate-binding domain/module (CBM), such as a starch binding domain, and optional a linker.

Specific examples of contemplated hybrid alpha-amylases include those disclosed in Table 1 to 5 of the examples in U.S. application No. 60/638,614, including Fungamyl variant with catalytic domain JA118 and Athelia rolfsii SBD (SEQ ID NO: 100 in U.S. application No. 60/638,614), Rhizomucor pusillus alpha-amylase with Athelia rolfsii AMG linker and SBD (SEQ ID NO:101 in U.S. application No. 60/638,614), Rhizomucor pusillus alpha-amylase with Aspergillus niger glucoamylase linker and SBD (which is disclosed in Table 5 as a combination of amino acid sequences SEQ ID NO. 20, SEQ ID NO: 72 and SEQ ID NO: 96 in U.S. application Ser. No. 11/316,535) or as V039 in Table 5 in WO 2006/069290, and Meripilus giganteus alpha-amylase with Athelia rolfsii glucoamylase linker and SBD (SEQ ID NO: 102 in U.S. application No. 60/638,614). Other specifically contemplated hybrid alpha-amylases are any of the ones listed in Tables 3, 4, 5, and 6 in Example 4 in U.S. application Ser. No. 11/316,535 and WO 2006/069290 (hereby incorporated by reference).

Other specific examples of contemplated hybrid alpha-amylases include those disclosed in U.S. Application Publication no. 2005/0054071, including those disclosed in Table 3 on page 15, such as Aspergillus niger alpha-amylase with Aspergillus kawachii linker and starch binding domain.

Contemplated are also alpha-amylases which exhibit a high identity to any of above mention alpha-amylases, i.e., more than 70%, more than 75%, more than 80%, more than 85% more than 90%, more than 95%, more than 96%, more than 97%, more than 98%, more than 99% or even 100% identity to the mature enzyme sequences.

An acid atpha-amyiases may according to the invention be added in an amount of 0.1 to 10 AFAU/g DS, preferably 0.10 to 5 AFAU/g DS, especially 0.3 to 2 AFAU/g DS or 0.001 to 1 FAU-F/g DS, preferably 0.01 to 1 FAU-F/g DS.

### Commercial Alpha-Amylase Products

Preferred commercial compositions comprising alpha-amylase include MYCOLASE from DSM, BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA, SPEZYME XTRA™ (Genencor Int., USA), FUELZYME™ (from Verenium Corp, USA); and the acid fungal alpha-amylase sold under the trade name SP288 (available from Novozymes A/S, Denmark).

### Carbohydrate-Source Generating Enzyme

The term “carbohydrate-source generating enzyme” includes glucoamylase (being glucose generators), beta-amylase and maltogenic amylase (being maltose generators). A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acidic fungal alpha-amylase activity (AFAU) per glucoamylase activity (AGU) (AFAU per AGU) may in an embodiment of the invention be at least 0.1, in particular at least 0.16, such as in the range from 0.12 to 0.50 or more or between 0.1 and 100, in particular between 2 and 50, such as in the range from 10-40.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al., 1984, EMBO J. 3 (5): 1097-1102), or variants thereof, such as those disclosed in WO 92/00381, WO 00/04136 and WO 01/04273 (from Novozymes, Denmark); the A. awarmori glucoamylase disclosed in WO 84/02921, A. oryzae glucoamylase (Agric. Biol. Chem., 1991, 55 (4): 941-949), or variants or fragments thereof. Other Aspergillus glucoamylase variants include variants with enhanced thermal stability: G137A and G139A (Chen et al., 1996, Prot. Eng. 9: 499-505); D257E and D293E/Q (Chen et al., 1995, Prot. Eng. 8: 575-582); N182 (Chen et al. 1994: Biochem. J. 301: 275-281); disulphide bonds, A246C (Fierobe et al. 1996, Biochemistry 35: 8698-8704; and introduction of Pro residues in position A435 and S436 (Li et al., 1997, Protein Eng. 10: 1199-1204.

Other glucoamylases include Athelia rolfsii (previously denoted Corticium rolfsii) glucoamylase (see U.S. Pat. No. 4,727,026 and Nagasaka et at., 1998, “Purification and properties of the raw-starch-degrading glucoamytases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamytases contemplated include glucoamytases from the genus Clostridium, in particular C. thermoamylolyticum (EP 135,138), and C. thermohydrosulfuricum (WO 86/01831) and Trametes cingulata disclosed in WO 2006/069289 (which are hereby incorporated by reference).

Also hybrid glucoamytase are contemplated according to the invention. Examples the hybrid glucoamylases disclosed in WO 2005/045018. Specific examples include the hybrid glucoamylase disclosed in Tables 1 and 4 of Example 1 (which hybrids are hereby incorporated by reference.).

Contemplated are also glucoamytases which exhibit a high identity to any of above mention glucoamylases, i.e., more than 70%, more than 75%, more than 80%, more than 85% more than 90%, more than 95%, more than 96%, more than 97%, more than 98%, more than 99% or even 100% identity to the mature enzymes sequences.

Commercially availabte compositions comprising glucoamylase include AMG 200L; AMG 300 L: SAN™, SUPER, SAN™, EXTRA L, SPIRIZYME™ PLUS, SPIRIZYME™ FUEL, SPIRIZYME™ B4U, SPIRIZYME™ ULTRA and AMG™ E (from Novozymes A/S); OPTIDEX™ 300, GC480™ and GC147™ (from Genencor Int., USA); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, especially between 1-5 AGU/g OS, such as 0.1-2 AGU/g OS, such as 0.5 AGU/g DS.

### Beta-Amylase

A beta-amylase (E.C 3.2.1.2) is the name traditionally given to exo-acting maltogenic amylases, which catalyzes the hydrolysis of 1,4-alpha-glycosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (Fogarty and Kelly, 1979, Progress in Industrial Microbiology 15; 112-115). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

### Maltogenic Amylase

The amylase may also be a maltogenic alpha-amylase. A “maltogenic alpha-amylase” (glucan 1,4-alpha-maltohydrolase, E.C 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

### Proteases

The protease may be any protease, such as of microbial or plant origin. In a preferred embodiment the protease is an acid protease of microbial origin, preferably of fungal or bacterial origin.

Suitable proteases include microbial proteases, such as fungal and bacterial proteases. Preferred proteases are acidic proteases, i.e., proteases characterized by the ability to hydrolyze proteins under acidic conditions below pH 7.

Contemplated acid fungal proteases include fungal proteases derived from Aspergillus, Macor, Rhizopus, Candida, Coriolus, Eridothia, Enthomophtra, Irpex, Penicillium, Sclerotium, and Torulopsis. Especially contemplated are proteases derived from Aspergillus niger (see. e.g., Koaze et al., 1964, Agr. Biol. Chem, Japan, 28: 216), Aspergillus saitoi (see, e.g., Yoshida, 1954, J. Agr Chem. Soc. Japan, 28: 66), Aspergillus awavmori (Hayashida et al., 1977, Agric. Biol. Chem. 42(5): 927-933, Aspergillus aculeatus (WO 95/02044), or Aspergillus oryzae, such as the pepA protease; and acidic proteases from Mucor pusillus or Mucor miehei.

Contemplated are also neutral or alkaline proteases, such as a protease derived from a strain of Bacillus. A particular protease contemplated for the invention is derived from Bacillus amyloliquefaciens and has the sequence obtainable at Swissprot as Accession No. P06832. Also contemplated are the proteases having at least 90% identity to amino acid sequence obtainable at Swissprot as Accession No. P06832 such as at least 92%. at least 95%, at least 96%, at least 97%, at least 98%, or particularly at least 99% identity.

Further contemplated are the proteases having at least 90% identity to amino acid sequence disclosed as SEQ ID NO: 1 in the WO 2003/048353 such as at 92%, at least 95%, at least 96%, at teast 97%, at least 98%, or particutarly at least 99% identity.

Also contemplated are papain-like proteases such as proteases within E.C. 3.4.22.* (cysteine protease), such as EC 3.4.22.2 (papain), EC 3.4.22.6 (chymopapain), EC 3.4.22.7 (asclepain), EC 3.4.22.14 (actinidain), EC 3.4.22.15 (cathepsin L), EC 3.4.22.25 (glycyl endopeptidase) and EC 3.4.22.30 (caricain).

In an embodiment the protease is a protease preparation derived from a strain of Aspergillus, such as Aspergillus oryzae. In another embodiment the protease is derived from a strain of Rhizomucor, preferably Rhizomucor mehei. In another contemplated embodiment the protease is a protease preparation, preferably a mixture of a proteolytic preparation derived from a strain of Aspergillus, such as Aspergillus oryzae, and a protease derived from a strain of Rhizomucor, preferably Rhizomucor mehei.

Aspartic acid proteases are described in, for example, Handbook of Proteolytic Enzymes, Edited by Barrett, Rawlings and Woessner, Academic Press, San Diego, 1998, Chapter 270). Suitable examples of aspartic acid protease include, e.g., those disclosed in Berka et al., 1990, Gene 96. 313; Berka et al., 1993, Gene, 125: 195-198; and Gomi et al., 1993. Biosci, Biotech. Biochem. 57: 1095-1100, which are hereby incorporated by reference.

The protease may be present in an amount of 0.0001-1 mg enzyme protein per g DS: preferably 0.001 to 0.1 mg enzyme protein per g DS. Alternatively, the protease may be present in an amount of 0.0001 to 1 LAPU/g OS, preferably 0.001 to 0.1 LAPU/g DS and/or 0.0001 to 1 mAU-RH/g DS, preferably 0.001 to 0.1 mAU-RH/g DS or in the amounts of 0.1-1000 AU/kg dm, preferably 1-100 AU/kg DS and most preferably 5-25 AU/kg DS.

### Use

In the third aspect the invention relates to the use of one or more of the compounds listed in the “Detoxifying compounds” section above, such as especially gallic acid or a amidase (e.g., the ones listed above), and anhydrase (e.g., the ones listed above) for detoxifying pre-treated lignocellulose-containing material.

The detoxification may be a separate or integral step in a fermentation product production process of the invention.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties,

### Materials & Methods

### Materials

Yeast Preparation: Freeze-dried RED STAR™ Ethanol Red yeast re-hydrated in 10×YP media for 30 minutes at 32° C. It was dosed into the fermentations at a dose of 0.2 g/L.

Gallic Acid: Sigma G7384—(3,4,5-trihydroxybenzoic acid)

Amidase: amidase from Pseudomonas aeruginosa (Sigma Product # A6691)

Carbonic Anhydrase: carbonic anhydrase from bovine erythrocytes (lyophilized powder, ≧2,500 W-A units/mg protein) Sigma Product # C3934

Cellulolytic Preparation A: Cellulolytic composition comprising a polypeptide having cellulolytic enhancing activity (GH61A) disclosed in WO 2005/074656; a beta-glucosidase (fusion protein disclosed in U.S. application No. 60/832,511), and cellulolytic enzymes preparation derived from Trichoderma reesei. Cellulase Preparation A is disclosed in U.S. application No. 60/941,251 (incorporated by reference).

### Methods

### Determination of Identity

The relatedness between two amino acid sequences or between two nucleotide sequences is described by the parameter “identity”.

The degree of identity between two amino acid sequences may be determined by the Clustal method (Higgins, 1989, CABIOS 5: 151-153) using the LASERGENE™, MEGALIGN™ software (DNASTAR, Inc., Madison, Wis.) with an identity table and the following multiple alignment parameters, Gap penalty of 10 and gap length penalty of 10. Pairwise alignment parameters are Ktuple=1, gap penalty=3, windows=5, and diagonals=5.

The degree of identity between two nucleotide sequences may be determined by the Wilbur-Lipman method (Wilbur and Lipman,. 1983, Proceedings of the National Academy of Science USA 80: 726-730) using the LASERGENE™ MEGALIGN™ software (DNASTAR, Inc,, Madison, Wis.) with an identity table and the following multiple alignment parameters Gap penalty of 10 and gap length penalty of 10. Pairwise alignment parameters are Ktuple=3, gap penalty=3, and windows=20.

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution, Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C. ±0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

Acid Alpha-Amylase activity (AFAU)

When used according to the present invention the activity of an acid alpha-amylase may be measured in FAU-F (Fungal Alpha-Amylase Unit) or AFAU (Acid Fungal Alpha-amylase Units).

### Determination of FAU-F

FAU-F Fungal Alpha-Amylase Units (Fungamyl) is measured relative to an enzyme standard of a declared strength.

Reaction conditions

| | |
|---|---|
| Temperature | 37° C. |
| pH | 7.15 |
| Wavelength | 405 nm |
| Reaction time | 5 min |
| Measuring time | 2 min |

A folder (EB-SM-0216.02) describing this standard method in more detail is available on request from Novozymes A/S, Denmark, which folder is hereby included by reference.

### Acid Alpha-Amylase Activity (AFAU)

Acid alpha-amylase activity may be measured in AFAU (Acid Fungal Alpha-amylase Units); which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3,2.1.1) hydrolyzes alpha-1,4-glycosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

$$STARCH + IODINE {{}_{40 ° , pH 2.5}}^{ALPHA \text{-} AMYLASE} DEXTRINS + OLIGOSACCHARIDES$$

$$λ = 590 nm$$

$$blue \text{/} violet \qquad t = 23 sec . \qquad decoloration$$

### Standard Conditions/Reaction Conditions:

| | |
|---|---|
| Substrate: | Soluble starch, approx. 0.17 g/L |
| Buffer: | Citrate, approx. 0.03 M |
| Iodine (I₂): | 0.03 g/L |
| CaCl₂: | 1.85 mM |
| pH: | 2.50 ± 0.05 |
| Incubation temperature: | 40° C. |
| Reaction time: | 23 seconds |
| Wavelength: | 590 nm |
| Enzyme concentration: | 0.025 AFAU/mL |
| Enzyme working range: | 0.01-0.04 AFAU/mL |

A folder EB-SM-0259.02/01 describing this analytical method in more detail is available upon request to Novozymes A/S, Denmark, which folder is hereby included by reference.

### Measurement of Cellulase Activity Using Filter Paper Assay (FPU Assay)

### 1. Source of Method

1.1 The method is disclosed in a document entitled “Measurement of Cellulase Activities” by Adney, B. and Baker, J., 1996, Laboratory Analytical Procedure, LAP-006, National Renewable Energy Laboratory (NREL). It is based on the IUPAC method for measuring cellulase activity (Ghose, T. K., Measurement of Cellulae Activities, 1987, Pure & Appl. Chem. 59; 257-268.

### 2. Procedure

2.1 The method is carried out as described by Adney and Baker, 1996, supra except for the use of a 96 well plates to read the absorbance values after color development, as described below.

2.2 Enzyme Assay Tubes:

2.2.1 A rolled filter paper strip (#1 Whatman; 1×6 cm; 50 mg) is added to the bottom of a test tube (13×100 mm).

2.2.2 To the tube is added 1.0 mL of 0.05 M Na-citrate buffer (pH 4.80).

2.2.3 The tubes containing filter paper and buffer are incubated 5 min. at 50° C. (±0.1° C.) in a circulating water bath.

2.2.4 Following incubation, 0.5 mL of enzyme dilution in citrate buffer is added to the tube. Enzyme dilutions are designed to produce values slightly above and below the target value of 2.0 mg glucose.

2.2.5 The tube contents are mixed by gently vortexing for 3 seconds.

2.2.6 After vortexing, the tubes are incubated for 60 mins. at 50° C. (±0.1° C.) in a circulating water bath.

2.2.7 Immediately following the 60 min. incubation, the tubes are removed from the water bath, and 3.0 mL of DNS reagent is added to each tube to stop the reaction. The tubes are vortexed 3 seconds to mix.

2.3 Blank and Controls

2.3.1 A reagent blank is prepared by adding 1.5 mL of citrate buffer to a test tube.

2.3.2 A substrate control is prepared by placing a rolled filter paper strip into the bottom of a test tube, and adding 1.5 mL of citrate buffer.

2.3.3 Enzyme controls are prepared for each enzyme dilution by mixing 1.0 mL of citrate buffer with 0.5 mL of the appropriate enzyme dilution.

2.3.4 The reagent blank, substrate control, and enzyme controls are assayed in the same manner as the enzyme assay tubes, and done along with them.

2.4 Glucose Standards

2.4.1 A 100 mL stock solution of glucose (10.0 mg/mL) is prepared, and 5 mL aliquots are frozen. Prior to use, aliquots are thawed and vortexed to mix.

2.4.2 Dilutions of the stock solution are made in citrate buffer as follows:
G1=1.0 mL stock+0.5 mL buffer 6.7 mg/mL 3.3 mg/0.5 mL

G2=0.75 mL stock+0.75 mL buffer=5.0 mg/mL=2.5 mg/0.5 mL

G3=0.5 mL stock+1.0 mL buffer=3.3 mg/mL=1.7 mg/0.5 mL

G4=0.2 mL stock+0.8 mL buffer=2.0 mg/mL=1.0 mg/0.5 mL

2.4.3 Glucose standard tubes are prepared by adding 0.5 mL of each dilution to 1.0 mL of citrate buffer.

2.4.4 The glucose standard tubes are assayed in the same manner as the enzyme assay tubes, and done along with them.

2.5 Color Development

2.5.1 Following the 60 min. incubation and addition of DNS, the tubes are all boiled together for 5 mins. in a water bath.

2.5.2 After boiling, they are immediately cooled in an ice/water bath.

2.5.3 When cool, the tubes are briefly vortexed, and the pulp is allowed to settle. Then each tube is diluted by adding 50 microL from the tube to 200 microL of ddH₂O in a 96-well plate. Each well is mixed, and the absorbance is read at 540 nm.

2.6 Calculations (examples are given in the NREL document)

2.6.1 A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.

2.6.2 A plot of glucose produced (mg/0.5 mL) vs. total enzyme dilution is prepared with the Y- axis (enzyme dilution) being on a log scale.

2.6.3 A line is drawn between the enzyme dilution that produced just above 2.0 mg glucose and the dilution that produced just below that. From this time it is determined the enzyme dilution that would have produced exactly 2.0 mg of glucose.

2.6.4 The Filter Paper Units/mL (FPU/mL) are calculated as follows: FPU/mL=0.37/enzyme dilution producing 2.0 mg glucose

### Protease Assay Method—AU(RH)

The proteolytic activity may be determined with denatured hemoglobin as substrate. In the Anson-Hemoglobin method for the determination of proteolytic activity denatured hemoglobin is digested, and the undigested hemotobin is precipitated with trichloroacetic acid (TCA). The amount of TCA soluble product is determined with phenol reagent, which gives a blue color with tyrosine and tryptophan.

One Anson Unit (AU-RH) is defined as the amount of enzyme which under standard conditions (i.e., 25° C., pH 5.5 and 10 min. reaction time) digests hemoglobin at an initial rate such that there is liberated per minute an amount of TCA soluble product which gives the same color with phenol reagent as one milliequivalent of tyrosine.

The AU(RH) method is described in EAL-SM-0350 and is available from Novozymes A/S Denmark on request.

### Proteolytic Activity (AU)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TCA soluble product is determined with phenol reagent, which gives a blue color with tyrosine and tryptophan.

One Anson Unit (AU) is defined as the amount of enzyme which under standard conditions (i.e., 25° C. pH 7.5 and 10 min. reaction time) digests hemoglobin at an initial rate such that there is liberated per minute an amount of TCA soluble product which gives the same color with phenol reagent as one milliequivalent of tyrosine.

A folder AF 4/5 describing the analytical method in more detail is available upon request to Novozymes A/S, Denmark, which folder is hereby included by reference.

### Protease Assay Method (LAPU)

1 Leucine Amino Peptidase Unit (LAPU) is the amount of enzyme which decomposes 1 microM substrate per minute at the following conditions: 26 mM of L-leucine-nitroanilide as substrate, 0.1 M Tris buffer (pH 8.0), 37° C., 10 minutes reaction time.

LAPU is described in EB-SM-0298.02/01 available from Novozymes A/S (Denmark) on request.

### Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma Mv 8378) substrate per ml of 0.1 M citrate buffer, pH 5.0 at 37° C. for 30 minutes.

### Amidase Unit Definition (Sigma Units)

One unit will convert 1.0 micromole of acetamide and hydroxylamine to acetohydroxamate and ammonia per min at pH 7.2 at 37 C.

### Carbonic Anhydrase Unit Definition (Sigma Units)

One Wilbur-Anderson (W-A) unit will cause the pH of a 0.02 M Trizma buffer to drop from 8.3 to 6.3 per min at 0° C. (One W-A unit is essentially equivalent to one Roughton-Booth unit.

### EXAMPLES

### Example 1

Pretreatment of Fully Unwashed Pretreated Corn Stover (fuwPCS)

Dilute acid steam exploded corn stover (PCS) was diluted with water and adjusted to pH 5.0 with NH₄OH. The total solids (TS) level was 15 wt.-%. This sample was then saccharified for 63 hours at 50° C. with Cellutolytic Preparation A. Penicillin was added at a rate of 1 gl/L also added prior to saccharification was citrate buffer at a rate of 50 mL of 1 M citrate buffer per 100 ml of substrate. Following the saccharification step, the sample was filtered via a 0.2 micron Nalgene vacuum fitter system (Product # 8-000043-0803) to remove the solids and used for fermentation. The fuwPCS was then pipetted into separate sterile, 15 mL conical centrifuge tubes containing a small CO₂ vent hole.

### Dosing (Gallic Acid)

The pH was adjusted to about 2 using H₂SO₄; gallic acid was dosed at concentrations of 2 mM (GaA-L) and at 10 mM (GaA-H). The gallic acid was prepared by sonicating 1.99 mg of garlic acid in 100 ml of de-ionized water. The broth was allowed to stand at 20° C. overnight and then readjusted to pH 5 using NaOH before adding yeast.

### Dosing (Amidase)

The dosing for amidase was carried out by treating with 5.6 Units/5 g substrate (AMD-L) and also 56 Units/5 g substrate (AMD-H). The amidase treated samples were brought to a pH of 7 with NaOH and allowed to sit in an oven at a temperature of 37° C. for 18 hours as a pre-treatment.

### Fermentation

Fermentations were carried out in sterile 15 mL conical plastic centrifuge tubes at 32° C. for 48 hours at pH 5.0. A total of 5 grams of sample was fermented for each treatment. Treatments were run in triplicate.

### Analytical

Fermentation samples were collected after 24 hours for the 0.2 g/L yeast dose and analyzed for acetic acid and ethanol using an Agilent HPLC System with an analytical BIO-RAD Aminex HPX-87H column and a BIO-RAD Cation H refill guard column.

### Results

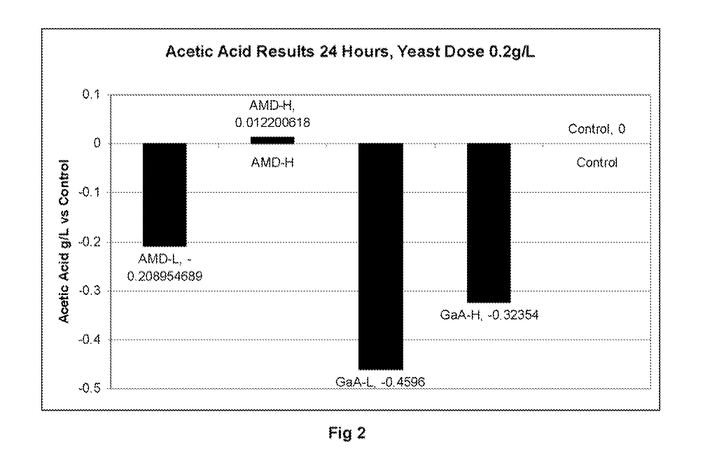

24 Hours Results. FIG. 1 shows the average ethanol results obtained for the yeast dose after 24 hours. Under these conditions, the level of ethanol obtained for the control samples is very low (about 2 g/L), suggesting that the inhibitors are negatively affecting the metabolism of the yeast. The results at 24 hours showed the amidase giving average yields of 21.8 g/L ethanol for the low dose and 23.6 g/L for the high dose. Gallic acid showed 19.4 g/L for the low dose and 17.8 g/L for the high dose. Gallic acid also showed a significant drop in the amount of acetic acid present in the fermentation (see FIG. 2).

### Example 2

### Carbonic Anhydrase and Amidase

### Pretreated Corn Stover Saccharification

Dilute acid steam exploded corn stover (PCS) was diluted with water and adjusted to pH 5.0 with NH₄OH. The total solids (TS) level was 16%. This sample was then saccharified for 72 hours at 50° C. with Cellulolytic Preparation A. Penicillin and citrate buffer were also added prior to saccharification. Following the saccharification step, the sample was filtered to remove the solids and the filtrate was used for fermentation. The fuwPCS was then pipetted into separate sterile, 15 mL conical centrifuge tubes containing a small CO₂ vent hole.

### Yeast Preparation

Freeze-dried RED STAR™ Ethanol Red yeast was re-hydrated in 10×YP media for 30 minutes at 32° C. it was dosed into the fermentations at a dose of 0.2 g/L.

### Dosing/Detoxification

Filtered, unwashed PCS was detoxified for 19 hours using the optimal conditions for each enzyme. For amidase, the pH of the fuwPCS was first adjusted up to 7.0 using NaOH, the amidase was added at the tested dosages, and the tubes were incubated at 37° C. The pH of the fuwPCS was then readjusted to 5.0 using H₂SO₄ prior to fermentation. For the carbonic anhydrase the pH was left at 5.0, the enzyme was added and the tubes were incubated at 37° C. All enzymes were diluted with de-ionized water prior to dosing. Dosing ranges for each enzyme were as follows in units/mL of the final solution for amidase and kilo units/mL for carbonic anhydrase.

| | | |
|---|---|---|
| Amidase | 7.0/37° C. | 0.3-1.1-5.4-16.1 (kilo units/mL) |
| | | 1.0-3.9-19.6-58.5 (units/g TS) |
| Carbonic Anhydrase | 5.0/37° C. | 0.7-3.3-16.6-33.3 (units/mL) |
| Anhydrase | | 4.9-24-121-243 (kilo units/g TS) |

### Fermentation

A total of 3.5 grams of sample was fermented for each treatment and the initial pH for all fermentations was 5.0. Treatments were run in triplicate. The final TS level was 13.7 wt.-%. These fermentations were run at a higher temperature of 37° C.

### Results

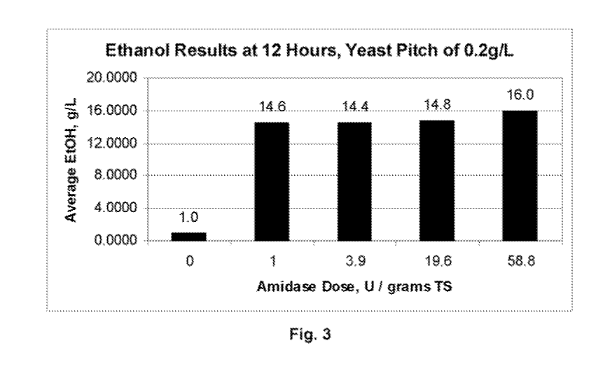

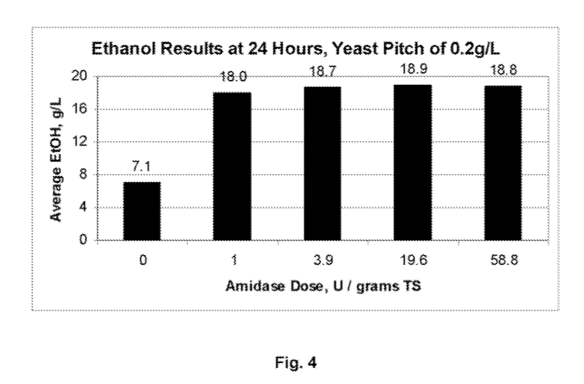

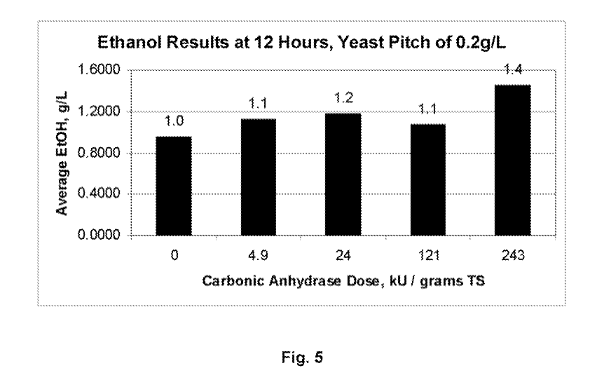

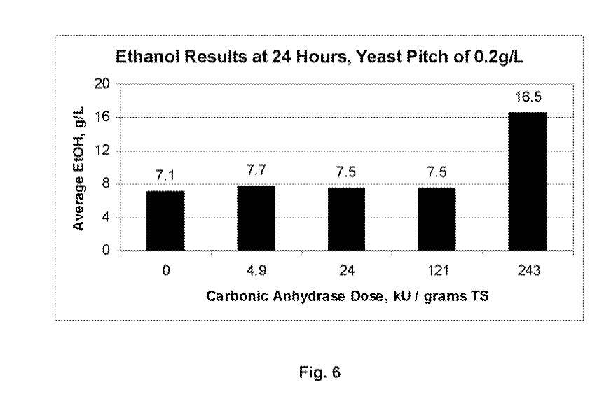

Fermentation samples were collected after 12 and 24 hours and analyzed for ethanol using an Agilent HPLC System with an analytical BIO-RAD Aminex HPX-87H column and a BIO-RAD Cation-H refill guard column. The results are displayed in FIGS. 3-6. The results for the amidase show a significant boosting effect on ethanol production by the yeast for all enzyme doses tested after both 12 and 24 hours of fermentation. The carbonic anhydrase results show significant boosting effects for the highest dose of the enzyme after both 12 and 24 hours of fermentation.

1-24. (canceled)

25. A process of detoxifying pre-treated lignocellulose-containing material, wherein pretreated lignocellulose-containing material is subjected to a compound selected from the group consisting of;
(a) compound capable of binding pre-treated lignocellulose degradation products;
(b) compound capable of binding acetic acid;
(c) amidase; and
(d) anhydrase; or a combination of two of more thereof.

26. The process of claim 25, wherein the detoxifying compound(s) is(are) capable of binding pre-treated lignin degradation products and/or hemicellulose degradation products.

27. The process of claim 26, wherein the hemicellulose is selected from the group consisting of xylan, galactoglucomannan, arabinogalactan, arabinoglucuronxylan, glucuronoxylan, and derivatives and combinations thereof.

28. The process of claim 25, wherein the amidase is selected from the group of Aminopeptidase B (EC 3.4.11.6), Cytosol alanyl aminopeptidase (EC 3.4.11.14), Dipeptidyl-peptidase II (EC 3.4.14.2), Dipeptidyl-peptidase III (EC 3.4.14.4), Dipeptidyl-peptidase IV (EC 3.4.14.5), Peptidyl-glycinamidase (EC 3.4.19.2), Omega-amidase (EC 3.5.1.3), Amidase (EC 3.5.1.4), Arylformamidase (EC 3.5.1.9), Penicillin amidase (EC 3.5.1.11). Aryl-acylamidase (EC 3.5.1.13), Aminoacylase (EC 3.5.1.14), Nicotinarmidase (EC 3.5.1.19), 5-aminopentanamidase (EC 3.5.1.30), Alkylamidase (EC 3.5.1.39), Acylagmatine amidase (EC 3.5.1.40), Formamidase (EC 3.5.1.49), Pentanamidase (EC 3.5.1.50), N-carbamoylputrescine amidase (EC 3.5.1.53), N,N-dimethylformamidase (EC 3.5.1.56), Tryptophanamidase (EC 3.5.1.57), N carbamoylsarcosine amidase (EC 3.5.1.59), 4-methyleneglutaminase (EC 3.5.1.67), D benzoylarginine-4-nitroanilide amidase (EC 3.5.1.72), Camitinamidase (EC 3.5.1.73), Arylalkyl acylamidase (EC 3.5.1.76), Glutathionylspermidine amidase (EC 3.5.1.78), Phthalyl amidase (EC 3.5.1.79), Mandelamide amidase (EC 3.5.1.86), L-lysine-lactamase (EC 3.5.2.11), Phosphoamidase (EC 3.9.1.1), N-sulfoglucosamine sulfohydrolase (EC 3.10.1.1), and Cyclamate sulfohydrolase (EC 3.10.1.2).

29. The process of claim 25, wherein the anhydrase is a carbonic anhydrase.

30. The process of claim 25, wherein the process is carded out at a pH below 7.

31. The process of claim 25, wherein the compound for detoxifying pretreated lignocellulose-containing material contains radicalizing hydroxy groups and an esterifiable carboxylic acid group.

32. The process of claim 25, wherein the detoxifying compound has a carboxylic acid group that can react with phenolic compounds from lignin and/or its degradation products.

33. The process of claim 25, wherein the detoxifying compound is gallic acid.

34. The process of claim 25, wherein a catalyst is present together with the detoxifying compound during detoxification.

35. A process of producing a fermentation product from lignocellulose containing material, comprising the steps of:
(a) pre-treating lignocellulose-containing material;
(b) detoxifying, wherein the detoxification is carried out in accordance with claim 25;
(c) hydrolyzing; and
(d) fermenting using a fermenting organism,

36. The process of claim 35, wherein one or more detoxifying compounds are added to the pre-treated lignocellulosic material in step (b).

37. The process of claim 35, wherein the detoxification in step (b) and the hydrolysis in step (c) are carried out simultaneously or sequentially.

38. The process of claim 35, wherein the detoxification in step (b) is carried separately and the hydrolysis in step (c) and the fermentation in step (d) are carried out simultaneously.

39. The process of claim 35, wherein all of steps (b), (c) and (d) are carried out simultaneously.

40. The process of claim 35, wherein the lignocellulose-containing material is chemically and/or mechanically pretreated in step (a).

41. The process of claim 35, wherein gallic acid, an amidase, and/or an anhydrase, is(are) used as detoxifying compound(s), preferably by addition before, during or after the pre-treatment in step (a).

42. The process of claim 35, wherein the hydrolysis in step (c) and the fermentation in step (d) are carried out simultaneously (SHF process) or sequentially (HHF process).

43. The process of claim 35, wherein the fermentation product is an alcohol preferably ethanol.

44. The process of claim 35, wherein a catalyst is present together with the detoxifying compound.
